(19) United States
(12) Patent Application Publication
(10) Pub. No.: US 2001/0041686 A1
(43) Pub. Date: Nov. 15, 2001

(54) Novel therapeutic application of enoxaparin

(76) Inventors: Veronique Mary, Organgis (FR); Jean-Marie Stutzmann, Villecresnes (FR); Andre Uzan, Paris (FR); Florence Wahl, Paris (FR)

Correspondence Address: AVENTIS PHARMACEUTICALS, INC., PATENTS DEPARTMENT, ROUTE 202-206, P.O. BOX 6800, BRIDGEWATER, NJ, 08807-0800, US

(21) Appl. No.: 09/752,926

(22) Filed: Jan. 2, 2001

Related U.S. Application Data
(60) Provisional application No. 60/188,352, filed on Mar. 9, 2000.

(30) Foreign Application Priority Data
Jan. 6, 2000 (FR) 0000137

(51) Int. Cl. A61K 31/727

(52) U.S. Cl. 514/56

(57) ABSTRACT

The present invention relates to the use of enoxaparin for the treatment of cerebral ischemia.

### CROSS REFERENCE TO RELATED APPLICATIONS

This application claims the benefit of U.S. Provisional Application No. 60/188,352, filed on Mar. 9, 2000, and of French Patent Application 00/00137. Filed on Jan. 6, 2000.

### FIELD OF THE INVENTION

The present invention relates to a novel therapeutic application of enoxaparin. More particularly, it relates to the use of enoxaparin to treat cerebral ischemias.

### BACKGROUND OF THE INVENTION

Enoxaparin (Lovenox®, Clexane®) is a low-molecular-weight heparin which is marketed for the prophylactic treatment of venous thromboembolic disease in moderate- or high-risk surgery, the prevention of coagulation in the extracorporeal circulation system during hemodialysis, the treatment of constituted deep venous thromboses and, in combination with aspirin, for the treatment of unstable angina and of acute non-Q wave myocardial infarction. Enoxaparin is also useful in the prevention and/or the treatment of trauma of the central nervous system (WO 98/53833) and of cerebral edemas (WO 98/53834).

Low molecular weight heparins have been tested in the prevention and/or treatment of deep venous thromboses in patients with cerebral ischemia but no effect on the ischemia has been shown (A. ELIAS et al., La Revue de Médecine Interne, 1, vol. XI, 95-98 (1990); M H. PRINS et al., Haemostasis, 19, 245-250 (1989); A G G. TURPIE et al., The Lancet, 523-526 (1987)).

### SUMMARY OF THE INVENTION

It has now been found that enoxaparin makes it possible to reduce cerebral ischemic sequelae and can thus be used for the treatment of cerebral ischemias.

### DETAILED DESCRIPTION OF THE INVENTION

This novel therapeutic use was determined in rats according to the following protocol:

Male Sprague Dawley rats (230-250 g Iffa Credo) are fed and watered ad libitum and kept in a light-dark cycle of 12 hours. Surgery was carried out under halothane (1.4% in a mixture of 70% N₂O/30% O₂). During anesthesia, normothermia is maintained by placing the rat under a thermostated cover. The left common carotid artery is isolated and a loose ligature is put in place. The left middle cerebral artery is exposed via a subtemporal craniotomy and a microclamp is placed in the proximal region of the artery, immediately followed by the ligation of the carotid artery. Two hours later, the animals are reanesthetized and the cerebral circulation is reestablished by removing the clamp from the middle cerebral artery and the ligature from the carotid artery. The rats are then placed in their cage in a thermostated room at 26-28° C.

48 hours after the surgery, a neurological examination is carried out for each rat by an examiner unaware of the treatment. The neurological scale used is described in Table 1.

TABLE 1

| Item | Normal | Deficit |
|---|---|---|
| Gripping reflex | 1 | 0 |
| right foreleg | | |
| Placing reaction | | |
| Loss of support | | |
| right foreleg | 1 | 0 |
| right hindleg | 1 | 0 |
| Righting reflex | | |
| Rotation | | |
| right side | 1 | 0 |
| left side | 1 | 0 |
| Abnormal postures | Absent | Present |
| Flexing of right foreleg | 1 | 0 |
| Twisting of the body | 1 | 0 |
| Overall neurological score | 7 | 0 |

After the neurological examination, the rats are humanely killed and their brains removed. 1.5 mm thick serial sections are prepared and stained with 2,3,5-triphenyltetrazolium chloride (TTC) at 2%. After 24 hours of post-fixing in a 10% formaldehyde solution, the lesion areas (cerebral infarct) are measured at the cortical and striatal levels; the volumes are calculated by integrating the lesioned surface areas. The values are expressed in mm³ (mean±S.E.M). Statistical analysis was carried out by a Mann-Whitney test or by a Kruskal-Wallis test for non-parametric variance analysis followed by a Dunn test for comparison between groups (*:p<0.05, **:p<0.01, ***:p<0.001 vs control group).

In Study 1, enoxaparin is administered to 12 rats at 1.5 mg/kg iv, 2 hours and 24 hours after the onset of the ischemia. A control group of 10 rats receives only the vehicle (physiological solution of sodium chloride at 0.9%) following the same protocol.

In Study 2, the therapeutic window of opportunity for enoxaparin is explored. The treatment starts 5 hours after the ischemia followed by a second administration at 24 hours. This study consists in an enoxaparin dose-effect on the cerebral lesions. The doses studied are 0.5:1 and 1.5 mg/kg iv (9-10 rats per group). A control group of 11 rats receives only the vehicle (physiological solution of sodium chloride at 0.9%).

In Study 3, enoxaparin is administered to 10 rats at 1.5 mg/kg iv, 5 and 24 hours after the onset of the ischemia. The control group of 13 rats receives only the vehicle (physiological solution of sodium chloride at 0.9%).

In Study 4, the protocol is the same as in the other studies but the left middle cerebral artery is permanently cauterized and no occlusion of the left carotid artery is performed. Enoxaparin is administered to 13 rats at 1.5 mg/kg iv, 5 and 24 hours after the onset of the ischemia. A control group of 13 rats receives only the vehicle (physiological solution of sodium chloride at 0.9%).

The results obtained are set forth in Table 2.

TABLE 2

| | | NEUROLOGICAL |
|---|---|---|
| | CORTICAL LESION | SCORE |
| STUDIES | (mm³) | 7-point scale |
| Study 1 | | |
| control group | 186 ± 18 | 1.7 ± 0.3 |
| enoxaparin group | 131 ± 13* | 3.1 ± 0.2** |
| 2 × 1.5 mg/kg iv | | |
| Study 2 | | |
| control group | 203 ± 12 | |
| enoxaparin group | | |
| 2 × 0.5 mg/kg iv | 164 ± 15 | |
| 2 × 1 mg/kg iv | 142 ± 24* | |
| 2 × 1.5 mg/kg iv | 129 ± 17* | |
| Study 3 | | |
| control group | 195 ± 12 | 1.8 ± 0.3 |
| enoxaparin group | 129 ± 16** | 3.4 ± 0.3*** |
| 2 × 1.5 mg/kg iv | | |
| Study 4 | | |
| control group | 137 ± 23 | 1.7 ± 0.2 |
| enoxaparin group | 71 ± 13* | 2.9 ± 0.3** |
| 2 × 1.5 mg/kg iv | | |

These results demonstrate that

in Study 1, enoxaparin significantly improves the neurological score 48 hours after the cerebral ischemia and, furthermore, significantly reduces the cortical lesion by 30%,

in Study 2, enoxaparin reduces the cortical lesion by 30% (2×1 mg/kg) and 36% (2×1.5 mg/kg),

in Study 3, enoxaparin significantly improves the neurological score and reduces the cortical lesion by 34%,

in Study 4, enoxaparin significantly improves the neurological score and reduces the cortical lesion by 49%.

No problem of bleeding was encountered during these studies.

The medicaments consist of enoxaparin in the form of a composition in which it is combined with any other pharmaceutically compatible product which may be inert or physiologically active. The medicaments according to the invention may be preferably used by the intravenous or subcutaneous route.

Sterile compositions for intravenous or subcutaneous administration are generally aqueous solutions. These compositions may also contain adjuvants, preferably selected from wetting, isotonizing, emulsifying, dispersing and stabilizing agents. The sterilization can be carried out in several ways, for example, by aseptisizing filtration, by incorporating sterilizing agents into the composition, or by irradiation. They may also be prepared in the form of sterile solid compositions which may be dissolved at the time of use in sterile water or any other injectable sterile medium.

As an example of a suitable composition, 20 mg of enoxaparin are dissolved in a sufficient quantity of distilled water to prepare 0.2 ml of solution.

The doses depend on the desired effect, the duration of the treatment and the route of administration used; they are generally between 0.2 mg and 4 mg/kg per day by the subcutaneous route, that is 14 to 280 mg per day for an adult with unit doses ranging from 5 to 280 mg.

In general, the doctor will determine the appropriate dosage according to the age, weight and all the other factors specific to the subject to be treated.

The present invention also relates to the method of treating cerebral ischemia in humans comprising the administration of an effective quantity of enoxaparin.

The present invention also relates to the use of enoxaparin for the preparation of a medicament which is useful for the treatment of cerebral ischemia.

1. A method for treating cerebral ischemia comprising administering to a patent in need thereof an effective amount of enoxaparin.

2. A composition for treating cerebral ischemia comprising an effective amount of enoxaparin and a pharmaceutically acceptable carrier.

3. The composition of claim 2 comprising 5 to 280 mg of enoxaparin per unit dose.
